(19) United States
(12) Patent Application Publication
(10) Pub. No.: US 2006/0257964 A1
(43) Pub. Date: Nov. 16, 2006

(54) METHOD OF MEASURING THE CONCENTRATION OF HYDROGEN PEROXIDE, PEROXYACETIC ACID, CHLORINATED COMPOUNDS AND OTHER AQUEOUS OXIDIZER COMPOUNDS

(75) Inventor: ROBERT A. LAROSE, MARLBOROUGH, CT (US)

Correspondence Address: ROBERT S. SMITH, 1131-0 TOLLAND TURNPIKE, SUITE 306, MANCHESTER, CT, 06040, US

(73) Assignee: BIOSAFE SYSTEMS L.L.C., GLASTONBURY, CT (US)

(21) Appl. No.: 10/908,518

(22) Filed: May 16, 2005

(51) Int. Cl. C12Q 1/30; C12M 1/34

(52) U.S. Cl. 435/27; 435/287.1

(57) ABSTRACT

In some embodiments of the apparatus detects the concentration of a substance selected from the group consisting of hydrogen peroxide, peroxyacetic acid, chlorinated compounds and other aqueous oxidizer compounds. The light source may be a laser, or light emitting diode. The source may be part of a transmitter and the detector may be part all of a receiver. The apparatus may further include a data logger cooperating with the light detector. The enzyme coated strip may be a strip coated with catalase and the the strip may be an inert paper. In some cases the enzyme coated strip changes color as a function of the concentration of the substance. The apparatus may further including fiber-optic cable cooperating with said light detector to detect transmitted light from the enzyme coated strip and a fiber-optic cable and/or a fiber optic cable cooperating with said source to direct light on the enzyme coated strip. The invention also includes the method for measuring the concentration of a substance which includes providing an enzyme coated strip, projecting a light beam on the enzyme coated strip, and detecting light from the enzyme coated strip.

### FIELD OF THE INVENTION

The invention primarily relates to the agriculture and horticulture fields and has particular application to measuring the concentration of a substance that is used as herbicide and/or fungicide. While the invention has particular application in these fields it will be understood that the invention has application in a myriad of other fields where it is desired to measure the concentration of hydrogen peroxide, peroxyacetic acid, chlorinated compounds and other aqueous oxidizer compounds. The invention may also be used to detect and measure other forms of oxidizing chemicals such as chlorine, chlorine dioxide, and bromine as well a ozone when they are applied by an aerosolized or vapor phase application method.

### BACKGROUND OF THE INVENTION

Peroxygen chemistry, both activated and non-activated, is increasingly being used to sanitize, disinfect and in some cases sterilize surfaces and environments. They can and do include the use of preferred liquid solutions of activated oxygen species are hydrogen peroxide, peracetic acid, sodium peroxide, potassium peroxide, calcium peroxide, potassium oxide and magnesium peroxide hydrogen peroxide. The preferred method includes generating atomized or aerosolized particles of peroxides with mechanical generators such as high and low pressure atomizing fogging systems, mechanical spinners, ultra sonic humidifiers, or by the use of injection into evaporative coolers, steam generation systems, or any other method in which to generate a liquid vapor of peroxide solutions than have a high oxidizing potential.

Aerosolized oxidizers may be used to fumigate areas with sanitizing concentrations of oxidizer compounds in order to either reduce the populations of microorganisms and insects or to sterilize surfaces of microorganisms, dormant spores, and insect larvae and in some cases adult insect populations.

Liquid oxidizer sanitizers such as stabilized hydrogen dioxide/peroxyacetic acid mixtures have produced good results. OxiDate™ broad-spectrum bactericide/fungicide, a leading product of this type, is manufactured by Biosafe Systems of Glastonbury, Conn. Biosafe Systems of Glastonbury, Conn. is also the assignee of this patent application. OxiDate™ broad-spectrum bactericide/fungicide can be used for disease control in potato storages post harvest.

Because the consequences of an inadequate distribution of the bactericide/fungicide may result huge crop losses and a substantial economic loss, the present invention has great application to ensuring adequate and substantially uniform distribution of the bactericide/fungicide in all such potato storage enclosures. While the invention has been described primarily in terms of potato storage it will be understood that the invention has broader application and other agriculture and horticulture fields.

The effectiveness of such techniques is a function of the concentration of the substance being utilized. In some cases, an insufficient concentration may not achieve the desired objective. In some cases, an excessive concentration may produce an undesired result. The utilization of mechanical generators such as high and low pressure atomizing fogging systems, mechanical spinners, ultra sonic humidifiers, or by the use of injection into evaporative coolers, steam generation systems, or any other method in which to generate a liquid vapor have conventionally required approximations based on the size of the room or chamber in which the vapor is being deposited, the rate of vapor production by the mechanical generator, and the time the generator is operating. Such approaches have not been wholly satisfactory.

Potato growers have used hydrogen dioxide sensitive strips placed on top of the piles to detect OxiDate™ broad-spectrum bactericide/fungicide. When the strips change color they stop the OxiDate™ broad-spectrum bactericide/fungicide application concluding that it has made it through the pile. Such approaches are useful; however, they are not adequate to ensure uniform distribution throughout huge storage facilities such as those that contain 20,000 tons of potatoes.

### SUMMARY OF THE INVENTION

It is an object of the present invention to provide a method in which to measure the concentration of aerosolized peroxides that is simple and accurate.

Another object the present invention is to provide a method that does not require the use of elaborate equipment.

A further object of the present invention is to facilitate treatment of enclosed area and monitoring the enclosed area to achieve a desired concentration.

Still another object of the invention is to provide an apparatus and method that is adaptable to very large storage facilities such as those that are capable of storing 20,000 tons of potatoes.

Yet another object to the invention is to provide an apparatus and method that will facilitate the storage of food products in a manner that will ensure much higher quality of the stored food product.

The invention may be used with respect to a process to sanitize airstreams that include microbes such as may be found in intake air used for heating and air conditioning systems (HAVC) wherein the process controls microorganisms in contaminated airsteams in a “kill on the fly method”.

The invention may be used to detect predetermined levels of oxidizers when they are used to sanitize any enclosed or unenclosed space to determine levels of concentrations of aerosoilized oxidizers.

It has now been found that these and other objects of the invention may be attained in apparatus for measuring the concentration of a substance which includes an enzyme coated strip, a light source projecting a light beam on the enzyme coated strip and a light detector detecting light from the enzyme coated strip.

In some embodiments of the apparatus detects the concentration of a substance selected from the group consisting of hydrogen peroxide, peroxyacetic acid, chlorinated, bromine and ozonated compounds and other aqueous oxidizer compounds. The light source may be a laser, or light emitting diode. The light detector may be selected from the group consisting of a photomultiplier tube, a photodiode, a phototransistor, a photodarlington, and photoconductive sensor. The source may be part of a transmitter and the detector may be part all of a receiver.

The apparatus may further include a data logger cooperating with the light detector. The enzyme coated strip may be a strip coated with catalase and the strip may be an inert paper. In some cases the enzyme coated strip changes color as a function of the concentration of the substance. The apparatus may further including fiber-optic cable cooperating with said light detector to detect transmitted light from the enzyme coated strip and a fiber-optic cable and/or a fiber optic cable cooperating with said source to direct light on the enzyme coated strip.

The invention also includes the method for measuring the concentration of a substance which includes providing an enzyme coated strip, projecting a light beam on the enzyme coated strip, and detecting light from the enzyme coated strip. The method may detect the concentration of a substance selected from the group consisting of hydrogen peroxide, peroxyacetic acid, chlorinated compounds and other aqueous oxidizer compounds.

The step of projecting a light beam may include utilizing a light source selected from a group that includes a light emitting diode and a laser. The step of detecting light includes utilizing a light detector that is selected from the group consisting of photomultiplier tube, a photodiode, a phototransistor and a photodarlington. In some cases the step of detecting light includes utilizing a detector that is part of a receiver and/or the step of projecting a light includes utilizing a source that is part of a transmitter. The method may also include utilizing a data logger coupled to the light detector. In addition, the step of providing an enzyme coated strip may include providing an enzyme coated strip that is coated with catalase.

The step of providing an enzyme coated strip may include providing an enzyme coating on an inert paper. This step may include using an enzyme coating that changes color as a function of the concentration of the oxidizer substance. In still other cases, the step of detecting light includes detecting light reflected from the strip and the step of detecting light includes

Another embodiment of the present invention includes apparatus for measuring the concentration of a substance in a food storage facility which includes a plurality of assemblies disposed at spaced intervals throughout a storage facility, each assembly including an enzyme coated strip; a light source disposed to project the a light beam on the it enzyme coated strip; and a light detector disposed to detect light from the enzyme coated strip.

This apparatus as may detect the concentration of a substance selected from the group consisting of hydrogen peroxide, peroxyacetic acid, chlorinated compounds and other aqueous oxidizer compounds and may a data logger. Some forms of the apparatus may further including apparatus for collecting and analyzing information from a plurality of assemblies including data loggers.

The invention also includes the method for measuring the concentration of a substance in a food storage facility which includes providing a plurality of assemblies disposed at spaced intervals throughout a storage facility wherein each assembly including an enzyme coated strip; a light source disposed to project the a light beam on the it enzyme coated strip; and a light detector disposed to detect light from the enzyme coated strip.

In some embodiments of this method, the method detects the concentration of a substance selected from the group consisting of hydrogen peroxide, peroxyacetic acid, chlorinated compounds and other aqueous oxidizer compounds and the method includes the step of providing an assembly includes providing a data logger as part of each assembly and may further include providing apparatus for collecting and analyzing information from a plurality of assemblies including data loggers.

### BRIEF DESCRIPTION OF THE DRAWING

The invention will better understood by reference to the accompanying drawing in which:

FIG. 1 is a diagrammatic representation of a retro-reflected light embodiment of the apparatus and method utilizing an enzyme test strip.

FIG. 2 is a diagrammatic representation of an elongated assembly incorporating three discrete assemblies, as shown in FIG. 1, disposed at different heights to permit use for measuring concentrations throughout the vertical extent of a storage facility which may contain potatoes stacked 20 feet deep.

FIG. 3 is a diagrammatic representation of a transmitted light embodiment of the apparatus in accordance with the present invention.

### DESCRIPTION OF THE PREFERRED EMBODIMENTS

An aqueous solution of activated peroxide may be used to fumigate a potato storage enclosure that contains stored potatoes. The humidified air is blown up from the floor of the potato storage facility and the peroxide solution is carried up through the pile of potatoes on air currents that are forced through the bottom of the pile, forcing the treated water particles to carry through the pile of potatoes, reaching the top. Most stored potatoes are stored in piles that may be twenty feet high. The apparatus and method of the present invention may be utilized at various levels of the potato pile in order to measure the concentrations of activated peroxide at the respective levels.

Peroxygen solutions may be injected into a moving HVAC (Heating, Ventilating and Air Conditioning) air stream in order to sanitize and treat the air stream. The invention may be used to measure the concentration of peroxides used. One or more peroxygen substance may be injected into a humidification systems used for a food storage enclosure. The invention may be used to maintain residual concentrations of ambient peroxygen to maintain a complete sanitary atmosphere.

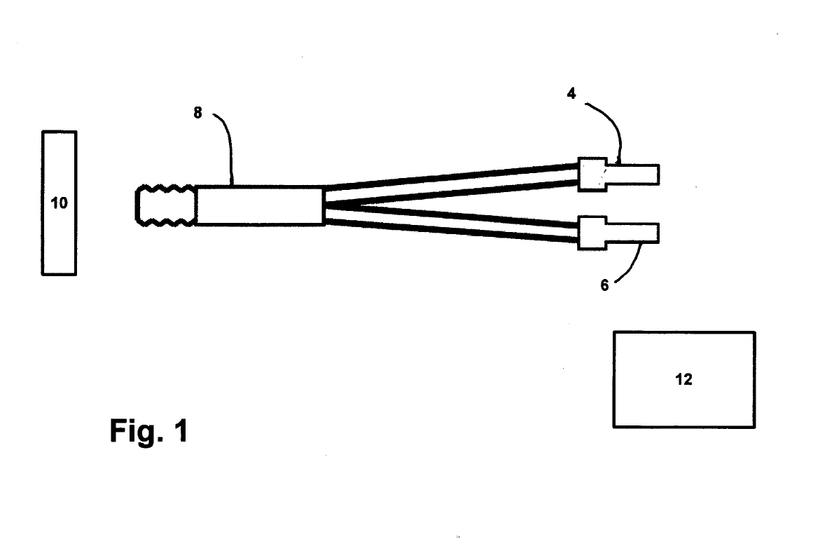

Referring now to FIG. 1, the method and apparatus in accordance with one form of the present invention measures in real time the concentration of hydrogen peroxide, peroxyacetic acid, chlorinated compounds and other aqueous oxidizer compounds that are introduced as a microaeorsol or liquid vapor. The invention utilizes the combination of a holder mounted enzyme test strip 10 that changes color in response to the presence of the specific substances together with fiber optic apparatus that senses the light reflected from the holder mounted enzyme test strip 10 to determine the concentration of the substance.

A holder mounted enzyme test strip 10 has an enzyme material deposited on a strip of chemically inert paper. The enzyme used is catalase. This enzyme reacts with hydrogen peroxide, More specifically, this enzyme reacts and changes color when exposed to concentrations of hydrogen peroxide, peroxyacetic acid, chlorinated compounds and other aqueous oxidizer compounds. The enzyme is responsive to various concentration ranges and will react with hydrogen peroxide within ranges of 100 pounds per minute to 1000 pounds per minute. The enzyme will turn a light shade of blue when exposed to concentrations of 100 pounds per minute but less than 200 pounds per minute. The enzyme will turn a darker shade of blue at concentrations of 200-300 pounds per minute and again another shade of blue from 300 pounds per minute to 600 pounds per minute and finally the darkest shade of blue from 700-1000 pounds per minute.

Fumigation of an enclosed room with 5000 pounds per minute (ppm) active ingredient (a. l.) of activated peroxygen solution with the express purpose of sterilizing all exposed surfaces within the room. The invention can be set as to turn off the fumigator and stop the treatment when the concentration of activated peroxide reaches a pre-determined level of 1000 pounds per minute active ingredient. Using OxiDate™ broad-spectrum bactericide/fungicide, as an example, this bactericide/fungicide contains 270,000 pounds per minute active ingredient. The bactericide/fungicide is mixed with water prior to application by a fogger or other apparatus in the ratio of 1:54. When a sensor at the point of application, for example a potato bin, indicates 1,000 pounds per minute active ingredient this correlates to an OxiDate™ bactericide/fungicide to water concentration of 1:270.

The holder mounted enzyme test strip 10 is exposed to a red light emitting diode (LED) light source located within a transmitter 4. The transmitter is a device that includes a source and driving electronics. It functions as an electrical-to-optical converter. The apparatus also includes a receiver 6. The receiver 6 is a terminal device that includes a detector and signal processing electronics. It functions as an optical-to-electrical converter. In the preferred embodiment, a bifurcated head or probe 8 that is branched to combine emitted light with received light in the same assembly. The bifurcated fibers may be equipped with a lens in the head or probe 8 for use in the retro-reflective mode as in the preferred embodiment of this invention. Commercially available products incorporate both the receiver 6 with a detector and the transmitter 4 with the light source in a single housing.

The LED light source or emitter, in some forms of the invention, is a light emitting diode. The source may also be a laser. The detector may be a photo detector, phototransistor or photo diode that is coupled to a fiber optic cable. Respective fiber optic conductors optically couple the detector and the source to an enzyme coated strip mounted in a holder 10. In this retro-reflective mode of the present invention, the respective ends (1) of the fiber optic conductor from the source in the transmitter 4 and (2) of the fiber optic conductor from the detector in the receiver 6, are positioned to respectively project light from the source on the holder mounted enzyme test strip 10 and transmit light from the holder mounted enzyme test strip 10 as shown in FIG. 1. The source end will project light to the holder mounted enzyme test strip 10 and the light will reflect back from the holder mounted enzyme test strip 10 to the detector in the receiver 6.

The receiver 6 produces a voltage that is a function of the light intensity that is reflected to the detector. The amount of reflected light back and the voltage produced is correlated to the amount of peroxide present.

A data logger 18 or other some type of recording device then reads the voltage. A data logger is an electronic instrument that records measurements (temperature, relative humidity, light intensity, on/off, open/closed, voltage, pressure and events) over time. Typically, data loggers are small, battery-powered devices that are equipped with a microprocessor, data storage and sensor. Most data loggers utilize turn-key software on a personal computer to initiate the logger and view the collected data. Data loggers are used in remote areas or anywhere you want the convenience of battery power to record measurements. They are ideal for those involved with field studies, transportation monitoring, HVAC tests, troubleshooting, quality studies, general research, and educational science. Hobbyists can even use data loggers since they are economically priced and easy-to-use. Data loggers can be used in a wide variety of applications. Their small size makes data loggers ideal for mounting in out-of-the-way locations. Typically, the data logger is connected to a personal computer. Then the turn-key software is used to select logging parameters (sampling intervals, start time, etc.) and initiate the logger. The logger is then disconnected and deployed in the desired location. The logger records each measurement and stores it in memory along with the time and date. The logger is then reconnected to the personal computer and the software is used again to display the data in tabular form as well as display graphically the measurements over time.

If the test strip turns a light shade of color, the a greater amount of light is reflected back and the corresponding amount of light is then converted over to a millivolt reading that would correspond into a chemical reading of say 500 to pounds per minute. If a test strip turns the darkest shade of color possible, then the least amount of light is reflected back. The low amount of reflected light is then processed and converted to a signal that records a high concentration of peroxides.

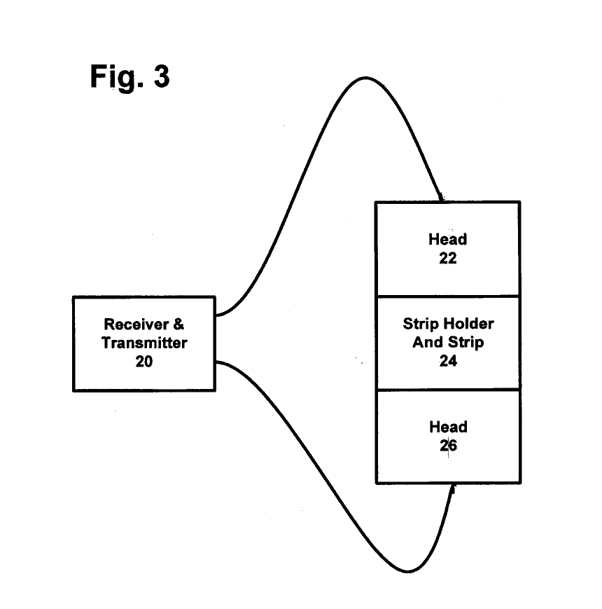

Referring now to FIG. 3 there is shown diagrammatically a light transmission embodiment of the present apparatus in method as opposed to the reflective embodiment described above. Instead of placing fiber optic light conductors on the same side of a holder mounted enzyme test strip 10 as in the reflection embodiment of the present invention, the embodiment illustrated in FIG. 3 illustrates a fiber optic receiver and transmitter assembly 20 that includes both the functional equivalent of the transmitter 4 including a light source and a receiver 6 that includes a detector. Respective fiber-optic cables connect the light source to a head 26 disposed on a first side of a holder mounted enzyme test strip 24 and the detector to a second side of the holder mounted enzyme test strip. The holder mounted enzyme test strip 24 is similar to the holder mounted enzyme test strip 10 except that the mounting allows transmission of light through the test strip instead of allowing light to be reflected from the test strip. Just as the color of the test strip affects the light that is reflected, the color of the township also affects the amount of light that will pass through the test strip.

Still other embodiments of the invention rely on light transmission through a cross linked polymer gel. Thus, in such embodiments the air gap is filled with a cross linked polymer gel, which changed color according to the amount of peroxides present, the amount of light transmitted through would also change. The detector will produce a voltage having a magnitude that is a function of the transmitted light. This voltage is then correlated to the amount of peroxides present.

The above description broadly describes the apparatus and method of the present invention. The following description describes in greater detail the method and apparatus of the present invention and particularly with respect to huge food storage installations such as those that store 20,000 tons of potatoes in one building.

Most table stock and processing potatoes are stored in environmentally controlled storage facilities. The storage facilities often hold potatoes stacked as high as 20 feet for many months. Storage diseases develop from potatoes infected in the field or in storage from pathogens in the soil residue attached to them at harvest. Many tons of potatoes are lost to diseases in storage every year. Researchers and growers have tried many things to lessen the losses but they remain severe. Protective fungicides sprayed onto the potatoes as they are conveyed into storage have met with limited success. Chemical sanitation with chlorine dioxide gas applied when the storage facility is loaded has also been unsuccessful. Liquid sanitizers in the form of stabilized hydrogen dioxide/peroxyacetic acid mixtures have produced good results. OxiDate™ broad-spectrum bactericide/fungicide, a leading product of this type, is manufactured by Biosafe Systems of Glastonbury, Conn. Biosafe Systems of Glastonbury, Conn. is also the assignee of this patent application. OxiDate™ broad-spectrum bactericide/fungicide can be used for disease control in potato storages post harvest. It has been used extensively in the United States for the past six years. The product is a federally registered product. OxiDate™ broad-spectrum bactericide/fungicide can be sprayed at load-in and or vaporized into the air forced up through the potatoes piled in storage. Some have argued that hydrogen dioxide/peroxyacetic acid mixtures will not move all the way through potato piles in the air stream but instead move to the surface by channeling up the sides of the piles. Potato growers have used hydrogen dioxide sensitive strips placed on top of the piles to detect OxiDate™ broad-spectrum bactericide/fungicide. When the strips change color they stop the OxiDate™ broad-spectrum bactericide/fungicide application concluding that it has made it through the pile. Testing using multiple the sensors at multiple elevations and multiple locations within multiple storage facilities has confirmed that the distribution of bactericide/fungicide concentration is substantially uniform throughout the storage facility. The process and method in accordance with the present invention evolved from testing intended to confirm the uniformity of the distribution of the bactericide/fungicide. Because the consequences of an inadequate distribution of the bactericide/fungicide may result in a substantial economic loss, the present invention has great application to ensuring adequate in substantially uniform distribution and all such potato storage enclosures. While the invention has been described primarily in terms of potato storage it will be understood that the invention has broader application and other agriculture and horticulture fields.

Photoelectric sensors detect objects with a modulated light beam that is either broken or reflected by the target. The sensor consists of a light source, a receiver to detect the emitted light and components to evaluate and amplify the detected signal. Common applications include area scanning, counting and detection of clear glass and plastics. Sensing modes for photoelectric sensors include through-beam, diffuse and retro-reflective.

Fiber optic sensors use a source, a receiver and a flexible cable packed with tiny fibers that transmit light. There are two types of fiber optic assemblies: individual and bifurcated. An individual fiber optic assembly guides light from a emitter to a location (such as a test strip), or from the location to the receiver. A bifurcated fiber optic assembly combines apparatus for emitting light with apparatus for receiving light in the same assembly. This fiber optic assembly may be joined to an optic amplifier by respective fiber optic cables.

Lasers can also be used as sensor light sources. These high-precision sensors have a high-intensity light, which makes setup and adjustment easy. The apparatus and method in accordance with a preferred embodiment of the invention utilizes a slim body analog fiber sensor model FX-11A manufactured by Sunx Limited Corporation Japan, 2431-1 Ushiyama-Cho Kasugai, Aichi Japan. This sensor has a fiber head or probe through which passes light from the sensor is projected on a surface being tested. The surface being tested reflects light back through the fiber head or probe. The reflected light received in the sensor causes the sensor to produce an analog voltage output of 1 to 5 VDC volts that varies with the intensity of the incident light. The sensor has an incident light indicator that that brightens in proportion to the amount of light returning to the sensor and a saturation light indicator that responds to the amount of light going out to the light probe.

Other apparatus used in the preferred embodiment includes a reflective Sunx fiber light probe FD-FM2 and fiber optic cable. Those still in the art will recognize that the analog fiber sensor model FX-11A manufactured by Sunx Limited Corporation Japan has four connections. They are: a first connection for a first fiber optic cable that cooperates with a source located in the sensor, a second connection for a second fiber-optic cable that cooperates with a receiver located in the sensor, a first cable for supplying an input voltage to the sensor and a second cable for delivering an output voltage that is a function of the magnitude of the light being sensed. A suitable data logger is marketed is the 15 VAC data logger marketed by MadgeTech, Inc., PO Box 50, 201 Route 103 West, Warner, N.H. 03278. Suitable detection papers are referred to in the industry as Peroxide 1,000 detection test papers having a functional range of 50-1,000 mg/l H₂O₂. Such papers are marketed by Biosafe Systems of Glastonbury, Conn. All such strips have chemical compositions on new unused strips that will be oxidized in the presence of an oxidizing agent. The oxidization that is produced causes a color change.

The system has 3 sensor assemblies. The assemblies consist of an analog light sensor, reflective light probe, data loggers, and mounting units (NEMA enclosure, PVC 4″ pipe, PVC sheet). The assemblies are wired in series and powered by a 24 VDC (20 amp) power supply. The light sensors convert electrical power to light which is transferred along fiber-optic cables to and from reflective light probes. The peroxide 1000 strips are positioned by a strip holder (not shown) in front of the light probe and reflect light that comes out one side of the probe back into the other side of it. The reflected light goes back to the sensor that produces a direct current output voltage that is proportional to the intensity of the light. The direct current output voltage from the sensor is sent to a battery powered data loggers at programmed intervals. The stored data can be down-loaded to data tables and viewed graphically using the software for the data loggers or it can also be saved to a Microsoft Excel spreadsheet with more robust graphing capabilities.

The system includes several provisions for calibration. The distance between the the light emitting end of the light probe and the test strip in the test strip holder is adjustable by mechanical means such as bolts and nuts. In addition, the intensity of the light beam from the source can be adjusted so that when the output of the source impacts a fresh unused test strip in the strip holder, the reflected light reaching the detector produces an output of the detector that is at the threshold of saturation (5V output) of the detector. These features allow the sensors to be calibrated very closely to the same starting output values whenever the strips are replaced with new ones. This is important to the precision and reliability of the system.

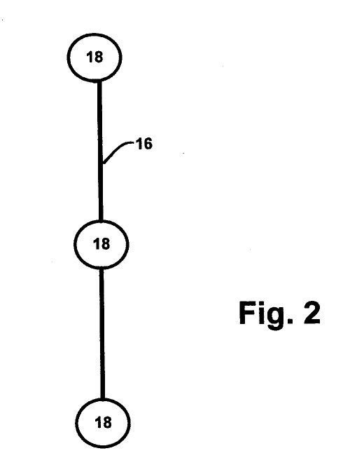

To establish the validity of the apparatus and methods in accordance with the invention a number of evaluation tests were conducted. Referring now to FIG. 2, there shown diagrammatically an elongated apparatus for insertion into a pile of potatoes. In the case of a pile that is 20 feet deep and the active ingredient is being supplied from the bottom of the pile is appropriate to monitor the concentration at 3 feet, 6 feet and 9 feet from the top of the pile. Accordingly the test utilized three sets of hardware. Each set of hardware included the components illustrated in FIG. 1. Thus, each set included a transmitter with a light source, receiver 6 with a detector, a bifurcated head 8, holder mounted enzyme test strip 10, and a data logger 12. As shown in FIG. 2 the elongated apparatus has one set disposed at 3 feet below the top surface of the pile, one set disposed at 6 feet below the top service of the pile and a third set disposed at 9 feet below the top service of the pile. For convenience in producing a diagrammatic representation the drawing implies that the entire structure of each set is located at each of these three nodes. Those skilled in the art will recognize that the data logger 12, transmitter 4, and receiver 6 are all disposed above of the top surface of the potatoes and that fiber-optic cables are to mention configured to extend down to the respective heads or probes 8. The three sensors were wired in series in the lab for this test.

The testing established substantial uniformity of the output voltage of the receiver 6 when the same sensor is used to measure reflected light from the surfaces of replicate test strips dipped in the same strength OxiDate™ bactericide/fungicide solution. Thus, the testing confirmed substantial uniformity despite possible variation in color intensity inherent in the strips as well as slight differences in test strip placement in the holder and adjustments to output voltage to blank strips prior to reading the replicate test

The testing further established substantial uniformity of the output voltage of the receiver 6 when connected to three different sensors each measuring reflected light from the surfaces of replicate test strips dipped in the same strength OxiDate™ bactericide/fungicide solutions. This test confirmed that the operational variability between respective sensors was acceptable.

Additional testing will also was performed to evaluate the range of output voltages of the receiver 6 throughout the range of the test strips. As noted above the range for the test strips with OxiDate™ bactericide/fungicide solutions is 100-1,000 ppm H₂O₂.

Additional testing was conducted utilizing a simulated pile of potatoes. In this test a 1:100 dilution of OxiDate™ bactericide/fungicide to tap water solution was vaporized and blown up through a 6 feet high column of potatoes and the amount of light reflected back from the surfaces of peroxide 1000 test strips in three sensors located at the top of the pile were measured over time. A tube, 6 feet long by 18″ wide, was used to contain 500 pounds of potatoes. A household vaporizer was used to apply the OxiDate™ bactericide/fungicide solution. It created the vapor through vibration and directed it through a self-contained fan. The unit vaporized 2 gallons of water in 12 hours. The sauna tube was placed on the floor of a wood frame which elevating it off the floor and supported it vertically. The vaporizer was placed below the frame the vapor was directed from the vaporizer to the base of the potato pile column. The tube was duct taped to the floor of the wood frame to force the vapor up through the tube. The 4″ PVC pipes housing the OxiDate™ bactericide/fungicide sensors were inserted about 6″ into the top of the potato pile. The unit ran until the peroxide 1000 strips darkened to their fullest extent. Voltage readings of the reflected light were recorded every minute for all three sensors. This test confirmed that the test strips would color from exposure to H₂O₂ vapor as opposed to dipping them in water as had been done in the tests described in the preceding three paragraphs. This test established at the sensors can be zeroed in calibrated effectively to give reliable and accurate readings.

Further confirmation of the validity of the method and apparatus was established by testing at an Idaho potato storage facility. The testing utilized the apparatus shown in FIG. 2. A plurality of these elongated assemblies, were positioned throughout a storage facility containing 5000 tons of potatoes. The elongate assemblies were spaced about 10-20 apart in a row running the long axis of the storage facility. Each sensor assembly included a 4 inch PVC pipe that was 6 feet long.

The field trial proved that OxiDate™ bactericide/fungicide moved through the pile since the sensor records show activity at first followed by the level then at the top as would be expected. The sensors dropped the expected 1.7V as predicted by both the lab and potato pile simulation. It can be concluded that the potatoes, even at the top of the potato pile were exposed to at least 1000 ppm a.i. of OxiDate™ bactericide/fungicide which is equivalent to a 1:270 dilution of OxiDate™ bactericide/fungicide to water.

Although the present invention has been described in terms of embodiments using a light source that is a laser or a light emitting diode, those skilled in the art will recognize that a wide variety of other light sources may be utilized in this apparatus. Examples include light emitting diodes (LEDS) including GaAIAs IR LED, GaAs IR LED, GaAsP Visible Red LE than Ds ; Solid State Semiconductor Lasers including GaAs (Hetrojunction) Lasers, GaAIAs (CW) Lasers, and Surface Emitting Lasers as well as externally excited solid state lasers, gas lasers fluorescent light sources including fluorescent lamps and cathode ray tubes (CRT) in addition to gas discharge sources including xenon gas discharge tubes, nitrogen gas (air) sparks and other gas discharge sources in addition to external light modulators. In addition, the present invention has been described in terms of a sensor having an analog output. Other embodiments of the invention may alternatively utilize a sensor having a digital output.

The respective methods and systems in accordance with the present system may utilize a computer that includes a microprocessor and memory and which cooperates with software that is commercially available or within the skill of practitioners in the programming arts.

Although the description above contains many specifics, these should not be construed as limiting the scope of the invention, but as merely providing illustrations of some of the presently preferred embodiments of this invention. Thus, the scope of this invention should be determined by the appended claims and their legal equivalents. Therefore, it will be appreciated that the scope of the present invention fully encompasses other embodiments which may become obvious to those skilled in the art, and that the scope of the present invention is accordingly to be limited by the appended claims, in which reference to an element in the singular is not intended to mean “one and only one” unless explicitly so stated, but rather “one or more” All structural, chemical, and functional equivalents to the elements of the above-described preferred embodiment that are known to those of ordinary skill in the art are expressly incorporated herein by reference and are intended to be encompassed by the present claims. Moreover, it is not necessary for a device or method to address each and every problem sought to be solved by the present invention, for it to be encompassed by the present claims. Furthermore, no element, component, or method step in the present disclosure is intended to be dedicated to the public regardless of whether the element, component, or method step is explicitly recited in the claims. No claim element herein is to be construed under the provisions of 35 U.S.C. 112, sixth paragraph, unless the element is expressly recited using the phrase “means for”.

1. Apparatus for measuring the concentration of a substance which comprises:
an enzyme coated strip;
a light source for projecting a light beam on said enzyme coated strip; and
a light detector detecting light from said enzyme coated strip.

2. The apparatus as described in claim 1 wherein the apparatus detects the concentration of a substance selected from the group consisting of hydrogen peroxide, peroxyacetic acid, chlorinated compounds, bromine compounds, ozonated compounds and other aqueous oxidizer compounds.

3. The apparatus as described in claim 1 wherein said light source is selected from the group consisting of a light emitting diode and a laser.

4. The apparatus as described in claim 1 wherein said light detector is a photomultiplier tube.

5. The apparatus as described in claim 1 wherein said light detector is selected from the group that consists of a photodiode, a phototransistor, a photodarlington, and a photoconductive sensor.

6. The apparatus as described in claim 1 wherein said source is part of a transmitter.

7. The apparatus as described in claim 1 wherein said light detector is part of a receiver.

8. The apparatus as described in claim 1 wherein said light detector is part of a receiver and said source is part of a transmitter.

9. The apparatus as described in claim 1 further including a data logger coupled to said light detector.

10. The apparatus as described in claim 1 wherein said enzyme coated strip is a strip coated with catalase.

11. The apparatus as described in claim 1 wherein said enzyme coated strip is an inert paper coated with an enzyme.

12. The apparatus as described in claim 1 wherein said enzyme coated strip changes color as a function of the concentration of the substance.

13. The apparatus as described in claim 1 further including a fiber optic cable cooperating with said light detector to detect transmitted light from said enzyme coated strip.

14. The apparatus as described in claim 1 further including a fiber optic cable cooperating with said source to direct light to said enzyme coated strip.

15. A method for measuring the concentration of a substance which comprises:
providing an enzyme coated strip;
projecting a light beam on the enzyme coated strip; and
detecting light from the enzyme coated strip.

16. The method as described in claim 15 wherein the method detects the concentration of a substance selected from the group consisting of hydrogen peroxide, peroxyacetic acid, chlorinated compounds and other aqueous oxidizer compounds.

17. The method as described in claim 15 wherein the step of projecting a light beam includes providing a light source that is selected from the group that consists of a light emitting diode and a laser.

18. The method as described in claim 15 wherein the step of detecting light from the strip includes providing a light detector that is selected from the group that includes a photomultiplier tube, a photodiode, a phototransistor,

19. The method as described in claim 15 wherein the step of detecting light includes utilizing a detector that is part of a receiver.

20. The method as described in claim 15 wherein the step of projecting a light includes utilizing a light source that is part of a transmitter.

21. The method as described in claim 15 wherein the step of detecting a light includes utilizing a detector that is part of a receiver.

22. The method as described in claim 15 wherein the step of projecting a light includes utilizing a light source that is part of a transmitter and the step of detecting a light includes utilizing a detector that is part of a receiver.

23. The method as described in claim 15 further including providing a data logger coupled to the light detector.

24. The method as described in claim 15 wherein the step of providing an enzyme coated strip includes providing an enzyme coated strip that is coated with catalase.

25. The method as described in claim 15 wherein the step of providing an enzyme coated strip includes providing an enzyme coating on an inert paper.

26. The method as described in claim 15 wherein the step of providing an enzyme coated strip includes providing an enzyme coated strip that changes color as a function of the concentration of the substance.

27-34. (canceled)

35. The method as described in claim 15 wherein the steps of providing an enzyme coated strip; projecting a light beam on the enzyme coated strip; and detecting light from the enzyme coated strip are performed in a food storage enclosure.

36. The method as described in claim 22 further including the step of providing a fiber optic cable cooperating with said light detector to detect transmitted light from said enzyme coated strip.
